(19) United States
(12) Patent Application Publication
(10) Pub. No.: US 2023/0226008 A1
(43) Pub. Date: Jul. 20, 2023

(54) Combination Verapamil and Mometasone Therapy for the treatment of Chronic Rhinosinusitis

(71) Applicant: Massachusetts Eye and Ear Infirmary, Boston, MA (US)

(72) Inventors: Benjamin S. Bleier, Weston, MA (US); Mansoor Amiji, Attleboro, MA (US); Maie Taha, Boston, MA (US)

(21) Appl. No.: 18/011,637

(22) PCT Filed: Jun. 21, 2021
(86) PCT No.: PCT/US2021/038281

§ 371 Date: Dec. 20, 2022

Related U.S. Application Data
(60) Provisional application No. 63/041,953, filed on Jun. 21, 2020.

(51) Int. Cl. A61K 31/277; A61K 31/58; A61K 45/06; A61P 11/02

(52) U.S. Cl. CPC A61K 31/277; A61K 31/58; A61K 45/06; A61P 11/02

(57) ABSTRACT

Described herein are methods for treating rhinosinusitis by administering to the subject an effective amount of verapamil and mometasone as well as compositions and kits for treating rhinosinusitis.

### CLAIM OF PRIORITY

This application claims the benefit of U.S. Provisional Application Ser. No. 63/041,953, filed on Jun. 21, 2020. The entire contents of the foregoing are incorporated herein by reference.

### TECHNICAL FIELD

Described herein are methods for treating rhinosinusitis by administering to the subject an effective amount of verapamil and mometasone as well as compositions and kits for treating rhinosinusitis.

### BACKGROUND

Chronic Rhinosinusitis (CRS) represents a spectrum of diseases unified by the presence of chronic inflammation of the sinonasal mucosa. Glucocorticoids (GC) have long been a mainstay in the treatment of CRS⁽¹⁻⁴⁾ with the goal of broad, non-targeted, suppression of the various inflammatory pathways leading to the clinical disease state. Mometasone furoate, in particular, has emerged as one of most favorable topical GCs due to its high potency and low bioavailability⁽⁵⁾. While generally effective⁽⁶⁻⁹⁾, it has been suggested that up to 50% of patients may demonstrate resistance to GCs⁽¹⁰⁾ for reasons which have yet to be fully elucidated. Consequently, the development of novel, cost effective, and targeted therapies represents a significant unmet need for patients with chronic rhinosinusitis.

### SUMMARY

Described herein are method of treating rhinosinusitis in a subject comprising identifying a subject having rhinosinusitis; and administering to the subject an effective amount of verapamil and mometasone.

Also described herein are methods of enhancing corticosteroid retention in a subject's sinonasal epithelial cells comprising: identifying a subject overexpressing P-gp in the subject's sinonasal epithelial cells; and administering to the subject an effective amount of verapamil and mometasone.

Also described herein are methods reducing inflammation in a subject's sinonasal epithelial cells comprising: identifying a subject overexpressing P-gp in the subject's sinonasal epithelial cells; and administering to the subject an effective amount of verapamil and mometasone.

In some embodiments, identifying a subject overexpressing P-gp in the subject's sinonasal epithelial cells comprises providing a sample comprising nasal secretions, preferably comprising nasal mucus, from a subject; determining a level of soluble p-glycoprotein (P-gp) in the sample; and comparing the level of P-gp in the sample to a reference level of P-gp; wherein a level of P-gp in the sample above the reference level indicates that the subject overexpresses P-gp.

In some embodiments, the subject has chronic rhinosinusitis (CRS) or CRS with nasal polyps (CRSwNP).

In some embodiments, the verapamil and mometasone are administered systemically.

In some embodiments, the verapamil and mometasone are administered locally to the subject's nasal passage and sinuses.

In some embodiments, the verapamil and mometasone are delivered to the subject's nasal passage and sinuses by nasal irrigation.

In some embodiments, the nasal irrigation is high volume, low positive pressure nasal irrigation.

In some embodiments, the verapamil and mometasone are delivered to the subject's nasal passage and sinuses by high volume, low positive pressure nasal irrigation with a saline solution. In some embodiments, the saline solution is an isotonic saline solution. In some embodiments, the saline solution is a hypertonic saline solution. In some embodiments, the hypertonic saline solution is about a 2% w/v saline solution.

In some embodiments, the verapamil and mometasone are administered to the subject as a verapamil and mometasone eluting implant placed in the subject's nasal passage or sinuses. In some embodiments, the implant is bioabsorbable.

In some embodiments, the verapamil is administered to the subject's nasal passage and sinuses by nasal irrigation and the mometasone is administered to the subject as a mometasone eluting implant placed in the subject's nasal passage or sinuses.

In some embodiments, the subject having rhinosinusitis was identified by endoscopy. In some embodiments, the subject having rhinosinusitis was identified by computed tomography. In some embodiments, the subject having rhinosinusitis was identified by observing the subject's symptoms and duration of symptoms.

In some embodiments, the method further comprises monitoring the efficacy of the treatment by endoscopy. In some embodiments, the method further comprises monitoring the efficacy of the treatment by computed tomography. In some embodiments, the method further comprises monitoring the efficacy of the treatment by observing the subject's symptoms and duration of symptoms. In some embodiments, the method further comprises surgically removing any nasal polyps present in the subject.

In some embodiments, the verapamil and mometasone are administered in combination with an antibiotic. In some embodiments, the antibiotic is selected from erythromycin or a pharmaceutically acceptable salt thereof, doxycycline or a pharmaceutically acceptable salt thereof, tetracycline or a pharmaceutically acceptable salt thereof, penicillin or a pharmaceutically acceptable salt thereof, beta-lactam or a pharmaceutically acceptable salt thereof, macrolide or a pharmaceutically acceptable salt thereof, fluoroquinolone or a pharmaceutically acceptable salt thereof, cephalosporin or a pharmaceutically acceptable salt thereof, and sulfonamide or a pharmaceutically acceptable salt thereof.

Also described herein are kits for treating rhinosinusitis in a subject comprising a pharmaceutical composition comprising an effective amount of verapamil and mometasone; and a device for delivering the pharmaceutical composition to the subject's nasal passage and sinuses. In some embodiments, the device delivers the pharmaceutical composition to the subject's nasal passage and sinuses in a liquid, nebulized, or aerosolized form. In some embodiments, the kit further comprises an antibiotic.

Also described herein are bioresorbable implants comprising verapamil and mometasone.

Also described herein are methods of preventing or treating chronic rhinosinusitis with nasal polyps in a subject, including administering to the subject a therapeutically effective amount of a P-glycoprotein inhibitor in combination with a therapeutically effective amount of momentasone. In some embodiments, the P-glycoprotein inhibitor can be, although is not limited to, Verapamil. In some embodiments, administration can be, although is not limited to, systemic, local or topical delivery. In some embodiments, local administration can be made to the subject's nasal passage and sinuses. In some embodiments, local or topical administration is directed to a polyp.

Also described herein are pharmaceutical compositions comprising a therapeutically effective amount of a Pglycoprotein inhibitor and a therapeutically effective amount of momentasone formulated with a pharmaceutically acceptable carrier for systemic delivery or local/topical adsorption. In some embodiments, the P-glycoprotein inhibitor can be, although is not limited to, Verapamil.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, “treatment” means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the compositions and methods of the present disclosure.

An “effective amount” is an amount sufficient to effect beneficial or desired results. For example, a therapeutically effective amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

The term “subject” is used throughout the specification to describe an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

The term “rhinosinusitis” as used herein includes acute and chronic rhinosinusitis, either with or without the presence of nasal polyps.

The term “about” a number as used herein refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F show that verapamil resulted in statistically significant intracellular retention of mometasone within high P-gp expressing tissues (e.g. nasal polyps) 1 hours following exposure relative to mometasone alone. This effect was not seen in low P-gp expressing tissues (e.g. inferior turbinates). FIGS. 1A and 1B show steroid retention in nasal polyps and turbinates; FIG. 1C shows within-group comparison of steroid retention in nasal polyps and turbinates; FIG. 1D shows within-group comparison of steroid retention in nasal polyps; FIGS. 1E and 1F show P-gp expression.

FIGS. 2A-2B shows the relative effect of verapamil on intracellular mometasone retention within nasal polyps was more pronounced when lower doses (FIG. 2A) of mometasone were utilized, compared to higher doses (FIG. 2B).

FIG. 3 shows the baseline intracellular retention of mometasone was strongly and significantly inversely correlated to P-gp expression within nasal polyps. This relationship was abrogated by inhibiting P-gp using verapamil.

FIGS. 4A-4C shows the combination of mometasone and verapamil significantly inhibited pro-inflammatory cytokine secretion from nasal polyps relative to vehicle control and either mometasone or verapamil given alone (* represents p=0.01 for IL-5 and IL-17, and p<0.001 for IL-6). FIG. 4A, IL-5; FIG. 4B, IL-6; FIG. 4C, IL-17.

FIG. 5 shows the results of an LDH assay.

FIGS. 6A shows a significant anti-inflammatory effect of combination mometasone and verapamil: inhibition of normalized (Day 2/Day 1) cytokine secretion among Type 2 (IL-5,6) and Type 17 (IL-17) cytokines in human nasal polyp explants (n=maximum of 8 patients). Among the type 2 cytokines, the combination of Verapamil (125 mcg/mL) and Mometasone (4.15 mcg/mL) significantly outperformed the anti-inflammatory effect of either medication alone relative to vehicle control (* p<0.05, **p<0.01, Student's ttest, error bars are SEM).

FIG. 6B shows a synergistic anti-inflammatory effect of combination mometasone and verapamil in type 2 inflammation: the total reduction from vehicle control in normalized (Day 2/Day 1) type 2 cytokine secretion (IL-5,6) from human nasal polyp explants following combination Verapamil (125 mcg/mL) and Mometasone (4.15 mcg/mL) treatment exceeded the additive effects of either medication alone.

FIGS. 7A-7B depict the structure of nasal polyps and explants.

FIGS. 8A-8E show that P-gp expression inversely correlated with mometasone retention. FIG. 8A: P-gp expression in nasal polyp explants (n=24, 4 explants from each of 6 patients) versus control turbinate explants (n=20, 3-4 explants from each of 3 patients). FIG. 8B: Mometasone tissue concentration after 60 min washout period. (c-d) Pearson correlation between P-gp expression and mometasone retention in nasal polyps following treatment with (FIG. 8C) mometasone alone (4.15 μg/mL), or (FIG. 8D) mometasone in combination verapamil (4.15 μg/mL and 125 μg/mL, respectively) after 60 min washout period. FIG. 8E P-gp levels within polyps with respect to the treatment condition demonstrating no change in treatment related expression. Data is presented as mean ±SEM. *, p<0.05 and **, p<0.01, unpaired two-tailed t-test.

FIGS. 9A-9B show that verapamil enhanced mometasone retention in organotypic polyp explants. Mometasone tissue concentrations in (FIG. 9A) polyp explants (minimum n=32, 4-6 explants from each of 6 patients) and (FIG. 9B) turbinate explants (minimum n=10, 5-6 explants from each of 2 patients), after 30 min exposure to either mometasone alone (4.15 μg/mL) or mometasone in combination with verapamil (4.15 μg/mL and 125 μg/mL, respectively), followed by washout. All data is presented as mean ±SEM. ns, non-significant and **, p<0.01, unpaired two-tailed t-test.

FIGS. 10A-10C show the dose response of mometasone retention in organotypic polyp explants. FIG. 10A: mometasone tissue concentration in polyp explants after 30 min exposure to either mometasone alone (2.075 μg/mL) or mometasone in combination with verapamil (2.075 μg/mL and 125 μg/mL, respectively), followed by 60 min washout (minimum n=20/group, 4-6 explants from each of 4 patients). (FIG. 10B) Relative fold change in verapamil (125 μg/mL) mediated mometasone retention in polyp explants by mometasone dose. (FIG. 10C) Mometasone retention in polyp explants (minimum n=4/group, 4-6 explants from 1 patient) upon co-treatment with 125, 250 or 500 μg/mL of verapamil for 30 min followed by 60 min washout period. All data is presented as mean ±SEM. ns, non-significant and *, p<0.05, Mann-Whitney test.

FIGS. 11A-11B show that verapamil significantly enhanced the anti-inflammatory effect of mometasone. FIG. 11A: histogram demonstrating normalized IL-5 secretion from organotypic nasal polyp explants in response to mometasone (4.15 μg/mL) or verapamil (125 μg/mL) treatments both in isolation and in combination. Only the combination of verapamil and mometasone significantly decreased IL-5 secretion relative to control (* p<0.05, Kruskal-Wallis test). FIG. 11B: LDH assay demonstrating lack of cytotoxicity within all conditions relative to vehicle control (BEGM). Day 1 represents 24 h incubation in BEGM+0.5 μg/mL of SEB and Day 2 BEGM+0.5 μg/mL of SEB+treatment condition.

### DETAILED DESCRIPTION

Within the past decade, there has been an increased focus on topical glucocorticoid (GC) therapeutic strategies for the management of CRS, both with and without Nasal Polyps (24-26). Mometasone furoate has garnered particular interest due to its unique pharmacochemistry. The addition of halogen and chloride at positions 9 and 21 increases the compound's affinity for the corticosteroid receptor and decreases its susceptibility to degradation while promoting hepatic metabolism (27). Simultaneously, it has been recognized that a subset of patients with CRS exhibit resistance to GCs (10) thereby limiting the efficacy of even the most potent molecules.

P-glycoprotein (P-gp) is a transmembrane efflux pump that utilizes ATP hydrolysis to transport a wide range of substrates across the plasma membrane. Prior studies have demonstrated that P-gp is locally overexpressed in the epithelium of CRS patients with type 2 inflammation (11, 12) and that it is capable of regulating epithelial secretion of multiple pro-inflammatory cytokines (13-15). Inhibition of P-gp improves prednisone retention in organotypic nasal polyp explants (16), raising the possibility that P-gp may participate in GC resistance through the active efflux of GC substrates. This phenomenon has been previously reported among steroid resistant patients with Crohn's disease who overexpress P-gp within their intestinal epithelium (17). P-glycoprotein has been recognized as a mechanism for clearing GCs from the cytoplasm however this effect is highly dependent on the specific amino acid moieties within the molecule (28). For example, dexamethasone, prednisolone and budesonide were found to have a high affinity for P-gp whereas triamcinolone acetonide did not (29). Despite these findings, no studies specifically examined whether mometasone furoate acted as a substrate for P-gp. Given the established presence of P-gp overexpression in type 2 endotypes of CRS (11, 12), we chose to study this phenomenon within a previously described organotypic nasal polyp explant model (13).

Using this approach, we first validated that within our sample set P-gp was overexpressed within nasal polyps as compared to healthy inferior turbinate controls. Inferior turbinate tissue was chosen on the basis of prior studies demonstrating minimal P-gp expression relative to sinus tissue; however, it is possible that use of a different control tissue could have impacted the results. We then demonstrated that the mean mometasone retention at 1 hour following exposure was statistically significantly reduced within nasal polyps as compared to low P-gp expressing inferior turbinates. While these results suggested mometasone was acting as a substrate for P-gp, we then confirmed this by correlating mometasone retention and P-gp expression within each individual explant. Using this approach, we found a 6-fold decrease in mometasone retention between the highest and lowest P-gp expressing explants, a value which would likely have significant implications for clinical efficacy.

We next sought to determine whether we could prevent this mometasone efflux by blocking P-gp activity. We elected to utilize verapamil given its established P-gp inhibitory activity (30) and its successful use in prior CRS clinical trials (23). We first demonstrated that the co-administration of verapamil abrogated the inverse relationship between P-gp expression and mometasone retention within nasal polyps. This effect translated into a significant increase in tissue mometasone concentration within polyps co-treated with verapamil relative to those treated with mometasone alone. Finally, our inflammatory assays confirmed that co-administration of mometasone with verapamil was superior with respect to reducing the canonical type 2 cytokine IL-5 relative to either drug alone. One of the limitations of our study is the non-specificity of verapamil as a P-gp inhibitor. Some of our observed results could be attributable to off target effects related to verapamil's calcium channel blocking activity in addition to its role in inhibiting P-gp.

The results of these studies have important clinical implications with regards to the topical treatment of CRS using mometasone. Based on prior reports, we can infer that the patients with more severe type 2 CRS endotypes will tend to have the highest levels of P-gp expression (12, 31). This study therefore suggests that these patients will also tend to be the most resistant to topical mometasone therapy. While verapamil has previously been shown to be effective in reducing both subjective and objective indices of CRSwNP as a monotherapy, this data indicates that verapamil also plays an important role in potentiating topical mometasone efficacy when given together.

Thus, provided herein are methods of treatment that comprise or consist of administering mometasone and verapamil in subjects with CRS or CRSwNP.

### P-gp Inhibitors

Methods using, and compositions comprising, P-gp inhibitors are described herein. Exemplary P-gp inhibitors are described herein. The methods may also use and the compositions may also comprise pharmaceutically acceptable salts, hydrates, solvates, prodrugs, stereoisomers, or tautomers of a P-gp inhibitors, e.g., a P-gp described herein.

### Verapamil

Verapamil hydrochloride is a calcium channel blocker which binds to the alpha subunit of L-type voltage dependent calcium (Cav1) channels thereby blocking the influx of calcium ions into the host cell (18). In addition to this function, verapamil was also one of the first inhibitors of P-gp to be identified in the 1980s (19). Several studies, including by our group (14), have reported that verapamil is capable of modulating inflammatory responses in human T-cells, animal models of asthma, and nasal polyps (18, 20-23) through its P-gp inhibitory function. Verapamil is 2-(3,4-dimethoxyphenyl)-5-[2-(3,4-dimethoxyphenyl)ethyl-methylamino]-2-propan-2-ylpentanenitrile:

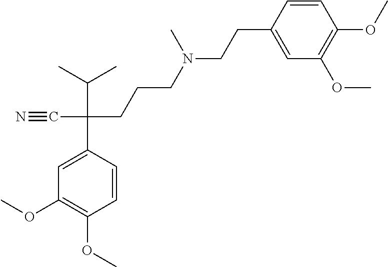

### Corticosteroids

Methods using, and compositions comprising, corticosteroids are described herein. Exemplary corticosteroids are described herein. The methods can also use, and the compositions can also comprise, pharmaceutically acceptable salts, hydrates, solvates, prodrugs, stereoisomers, or tautomers of a corticosteroid, e.g., a corticosteroid described herein.

### Mometasone and Mometasone Furoate

In some embodiments, the corticosteroid is mometasone. Mometasone ((8S,9R,10S,11S,13S,14S,16R,17R)-9-chloro-17-(2-chloroacetyl)-11,17-dihydroxy-10,13,16-trimethyl-6,7,8,11,12,14,15,16-octahydrocyclopenta[a]phenanthren-3-one) is a synthetic topical glucocorticoid receptor (GR) agonist with anti-inflammatory, anti-pruritic and vasoconstrictive properties that has the structure shown below.

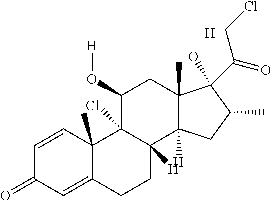

In some embodiments, the mometasone is mometasone furoate. Mometasone Furoate ([(8S,9R,10S,11S,13S,14S,16R,17R)-9-chloro-17-(2-chloroacetyl)-11-hydroxy-10,13,16-trimethyl-3-oxo-6,7,8,11,12,14,15,16-octahydrocyclopenta[a]phenanthren-17-yl]furan-2-carboxylate) is the furoate ester form of mometasone that has the structure shown below.

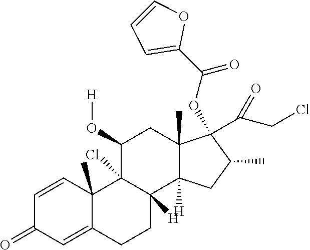 [SMILES: C[C@@H]1C[C@H]2[C@@H]3CCC4=CC(=O)C=C[C@]4(C)[C@@]3(Cl)[C@@H](O)C[C@]2(C)[C@@]1(OC(=O)c1ccco1)C(=O)CCl]

### Antibiotics

Methods using, and compositions comprising, antibiotics are described herein. Exemplary antibiotics are described herein. The methods may also use, and the compositions may also comprise, pharmaceutically acceptable salts, hydrates, solvates, prodrugs, stereoisomers, or tautomers of an antibiotic, e.g., an antibiotic described herein.

In some embodiments, the antibiotic is erythromycin.

In some embodiments, the antibiotic is doxycycline.

In some embodiments, the antibiotic is tetracycline.

In some embodiments, the antibiotic is penicillin.

In some embodiments, the antibiotic is a beta-lactam antibiotic.

In some embodiments, the antibiotic is a macrolide (i.e. a macrocyclic lactone with a ring of twelve or more members).

In some embodiments, antibiotic is fluoroquinolone.

In some embodiments, the antibiotic is a sulfonamide.

In some embodiments, the method using or composition comprising an antibiotic uses or comprises a combination of antibiotics, e.g., a combination of antibiotics described herein.

### Treatment

In the methods described herein, a subject with rhionsinusitis, e.g., chronic rhinosinusitis, e.g., CRSwNP, is administered a therapeutically effective amount of a P-gp inhibitor, e.g., verapamil, and a corticosteroid, e.g., mometasone. In some embodiments, the P-gp inhibitor and corticosteroid are administered in a single composition. In some embodiments, the P-gp inhibitor and corticosteroid are administered in separate compositions.

In some embodiments, a subject having rhinosinusitis, e.g., chronic rhinosinusitis (CRS) is identified and treated by administration to the subject an effective amount of a P-gp inhibitor, e.g., verapamil, and a corticosteroid, e.g., mometasone.

CRSwNP is Chronic Rhinosinusitis with Nasal Polyps whereas the term Chronic Rhinosinutis (CRS) encompasses patients with and without nasal polyps. In some embodiments, the present methods are used to treat subjects with CRS without nasal polyps, as some patients with CRS but without polyps still have polyp-like inflammation.

The subject having rhinosinusitis, e.g., chronic rhinosinusitis, may be identified by one of skill in the art based on known methods, e.g., based on detection of the presence of symptoms, by endoscopy, or by computed tomography. The efficacy of the treatment may be monitored by methods known in the art, e.g., by monitoring symptoms, by endoscopy or computed tomography. Improvements of the subject include a better symptom score, e.g. a better SNOT-22 or VAS score; a reduction in inflammation or nasal polyp burden as revealed by endoscopy, e.g. a better Lund-Kennedy score; or a reduction in mucosal thickening or sinus opacification as revealed by computed tomography (CT), e.g. a better Lund-Mackay score. The 22-item Sinonasal Outcomes Test (SNOT-22) is a questionnaire encompassing 22 major symptoms on rhinosinusitis and nasal polyps, and serves as a valuable tool to measure the severity of a subject's symptoms and their impact on health-related quality of life (Quintanilla-Dieck, et al., International Forum of Allergy & Rhinology 2012; 2(6):437-443). The SNOT-22 assessed 12 nasal- and sinus-related symptoms (nasal blockage, loss of sense of taste and smell; need to blow nose, sneezing, runny nose, cough, postnasal discharge, thick nasal discharge, ear fullness, dizziness, ear pain, and facial pain/pressure) and 10 psychological and behavioral symptoms (difficulty falling asleep, waking up at night, lack of a good night's sleep, waking up tired, fatigue, reduced productivity, reduced concentration, frustrated/restless/irritable, sad, and embarrassed) with participants scoring each symptom on a scale of 0 (absent) to 5 (severe) on average for the last week, for a total score range of 0 to 100. The SNOT-22 score is the mean for the 22 scores (Piccirillo et al., Otolaryngol Head Neck Surg 2002; 126:41-47). The 10-symptom visual analog (VAS) scale is a questionnaire based on the major and minor symptom diagnostic criteria for CRS as described by the American Academy of Otolaryngology—Head and Neck Surgery TFR. The VAS assessed subject-reported severity of each of the following symptoms on average experienced during the prior week: nasal drainage of pus, nasal obstruction/congestion, impaired sense of smell, facial pressure/pain, headache, bad breath, weakness/fatigue, dental pain, ear fullness/pain, and cough (Ryan, et al., Laryngoscope 2011; 121:674-678). The Lund-Kennedy endoscopy scoring system quantifies the pathologic states of the nose and paranasal sinuses as assessed by nasal endoscopy, focusing on the presence of polyps, discharge, edema, scarring or adhesions, and crusting (Ryan, et al., 2011). The Lund Mackay CT scoring system is the most widely used CT grading system for chronic rhinosinusitis. This scoring system consists of a scale of 0-2 dependent on the absence (0), partial (1) or complete (2) opacification of the sinus system and the osteomeatal complex as assessed by CT imaging (Hopkins et al., Otolaryngology—Head and Neck Surgery 2007; 137:555-561).

In some embodiments, the subject having rhinosinusitis, e.g., chronic rhinosinusitis, is identified by the presence and/or level of P-gp, e.g., as described in WO2014106021 or WO2017123933A1, which are hereby incorporated by reference in their entirety. In some embodiments, the efficacy of the treatment may be monitored by the presence and/or level of P-gp, e.g., as described in WO2014106021 or WO2017123933A1, which are hereby incorporated by reference in their entirety. Improvements of the subject include a reduction in the amount of secreted P-gp in a sample after treatment as compared to before treatment.

In some embodiments, a subject with rhinosinusitis is treated with the P-gp inhibitor, e.g., verapamil, and a corticosteroid, e.g., mometasone, in combination with other conventional treatments, e.g., antibiotics, to potentiate the effect of treatment. In some embodiments, the antibiotic is selected from the group consisting of a macrolide, e.g., erythromycin; a penicillin, e.g., amoxicillin, beta-lactam, ampicillin; a tetracycline, e.g., doxycycline, tetracycline; a sulfonamide, e.g. mafenide, sulfacetamide; a fluoroquinolone; a cephalosporin, e.g., ceftaroline fosamil, ceftobiprole; and combinations thereof.

In some embodiments, when a subject with rhinosinusitis has nasal polyps, surgical removal of such nasal polyps and/or sinus surgery can be performed in addition to administration of the P-gp inhibitor, e.g., verapamil, and a corticosteroid, e.g., mometasone, to the subject. Thus, a subject with rhinosinusitis may undergo both surgery and treatment with the P-gp inhibitor, e.g., verapamil, and a corticosteroid, e.g., mometasone, using the present methods.

### Pharmaceutical Compositions and Methods of Administration

The methods described herein include the use of pharmaceutical compositions comprising or consisting of a P-gp inhibitor, e.g., verapamil, and a corticosteroid, e.g., mometasone, as active ingredients.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. The P-gp inhibitor and corticosteroid (and optional antibiotic) can be provided in a concentrated form, along with salts to provide an isotonic (normal saline) solution and/or a hypertonic saline solution for comfortable nasal irrigation upon addition of water (e.g., distilled or other clean water, not necessarily sterile). In some embodiments, the salts comprise sodium chloride and a buffering agent, e.g., sodium bicarbonate, e.g., sufficient sodium chloride to provide a final concentration of 0.8-1%, e.g., 0.9 percent sodium chloride, and buffering agent to provide a pH of 4.5 to 7.

Pharmaceutical compositions are typically formulated to be compatible with its intended route of administration. Examples of routes of administration include nasal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for nasal application can include the following components: a diluent such as water, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

In some embodiments, the P-gp inhibitor, e.g., verapamil, and the corticosteroid, e.g., mometasone, are administered locally to the subject's nasal passage and sinuses by irrigation with a composition comprising the P-gp inhibitor, e.g., verapamil and the corticosteroid, e.g., mometasone. In some embodiments, the composition further comprises sodium chloride.

Nasal irrigation (also sometimes referred to as nasal douche, wash, or lavage) is a procedure that rinses the nasal cavity with liquid, e.g., isotonic or hypertonic saline solutions.

In some embodiments, the nasal irrigation is low positive pressure nasal irrigation. See, e.g., Pynnonen et al., “Nasal Saline for Chronic Sinonasal Symptoms,” Arch Otolaryngol Head Neck Surg 133(11):1115-20 (2007); Chong et al., “Saline Irrigation for Chronic Rhinosinusitis (Review),” Cochrane Database of Systematic Reviews 4:CD011995 (2016). Nasal irrigation can be performed with, for example, a low positive pressure from a spray bottle, a pump, a squirt bottle, a nebulizer, a gravity-based pressure using a vessel with a nasal spout, or an exhalation delivery system. Suitable devices include, for example, the Sinus Rinse product (NeilMed® Pharmaceuticals, Inc., Santa Rosa, Calif.) (see, e.g., U.S. Pat. Nos. 6,520,374 and 6,669,059 and US Trade Dress No. 3,559,683), a Neti Pot such as the NeilMed® NasaFlo® Neti Pot (see, e.g, U.S. Pat. No. 9,623,170B), and the Optinose® Exhalation Delivery Systems (see, e.g., WO2013124492A1).

In some embodiments, the composition comprising or consisting of the P-gp inhibitor, e.g., verapamil and the corticosteroid, e.g., mometasone, and sodium chloride is isotonic. In some embodiments, the composition comprising the P-gp inhibitor, e.g., verapamil and the corticosteroid, e.g., mometasone, and sodium chloride is hypertonic.

In some embodiments, the composition comprising or consisting of the P-gp inhibitor, e.g., verapamil and the corticosteroid, e.g., mometasone, and sodium chloride comprises about 0.9% (w/v) sodium chloride. In some embodiments, the composition comprising the P-gp inhibitor, e.g., verapamil and the corticosteroid, e.g., mometasone, and sodium chloride comprises about 2% (w/v) sodium chloride.

In some embodiments, the composition comprising or consisting of the P-gp inhibitor, e.g., verapamil and the corticosteroid, e.g., mometasone, and sodium chloride comprises from or from about 8.0 g/L to or to about 10.0 g/L sodium chloride. In some embodiments, the composition comprising the P-gp inhibitor, e.g., verapamil and the corticosteroid, e.g., mometasone, and sodium chloride comprises from or from about 8.5 g/L to or to about 9.5 g/L sodium chloride. In some embodiments, the composition comprising the P-gp inhibitor, e.g., verapamil and the corticosteroid, e.g., mometasone, and sodium chloride comprises 9.0 g/L or about 9.0 g/L sodium chloride.

In some embodiments, the composition comprising or consisting of the P-gp inhibitor, e.g., verapamil and the corticosteroid, e.g., mometasone, and sodium chloride comprises or consisting of from or from about 16.0 to or to about 20.0 g/L sodium chloride. In some embodiments, the composition comprising the P-gp inhibitor, e.g., verapamil and the corticosteroid, e.g., mometasone, and sodium chloride comprises from or from about 17.0 to or to about 19.0 g/L sodium chloride. In some embodiments, the composition comprising the P-gp inhibitor, e.g., verapamil and the corticosteroid, e.g., mometasone, and sodium chloride comprises 18 g/L or about 18 g/L sodium chloride.

In some embodiments, the composition comprising or consisting of the P-gp inhibitor, e.g., verapamil and the corticosteroid, e.g., mometasone, and sodium chloride comprises from or from about 4.0 g/L to or to about 20.0 g/L sodium chloride. In some embodiments, the composition comprising the P-gp inhibitor, e.g., verapamil and the corticosteroid, e.g., mometasone, and sodium chloride comprises from or from about 4.0 to or to about 18.0, from or from about 4.0 to or to about 16.0, from or from about 4.0 to or to about 14.0, from or from about 4.0 to or to about 12.0, from or from about 4.0 to or to about 10.0, from or from about 4.0 to or to about 8.0, from or from about 4.0 to or to about 6.0, from or from about 6.0 to or to about 20.0, from or from about 6.0 to or to about 18.0, from or from about 6.0 to or to about 16.0, from or from about 6.0 to or to about 16.0, from or from about 6.0 to or to about 14.0, from or from about 6.0 to or to about 12.0, from or from about 6.0 to or to about 10.0, from or from about 6.0 to or to about 8.0, from or from about 8.0 to or to about 20.0, from or from about 8.0 to or to about 18.0, from or from about 8.0 to or to about 16.0, from or from about 8.0 to or to about 14.0, from or from about 8.0 to or to about 12.0, from or from about 8.0 to or to about 10.0, from or from about 10.0 to or to about 20.0, from or from about 10.0 to or to about 18.0, from or from about 10.0 to or to about 16.0, from or from about 10.0 to or to about 14.0, from or from about 10.0 to or to about 12.0, from or from about 12.0 to or to about 20.0, from or from about 12.0 to or to about 18.0, from or from about 12.0 to or to about 16.0, from or from about 12.0 to or to about 14.0, from or from about 14.0 to or to about 20.0, from or from about 14.0 to or to about 18.0, from or from about 14.0 to or to about 16.0, from or from about 16.0 to or to about 20.0, from or from about 16.0 to or to about 18.0, from or from about 18.0 to or to about 20.0 g/L sodium chloride.

In some embodiments, the irrigation is with a high volume of liquid, e.g., 50 ml or more, e.g., 100 or 150 ml up to 250 or 300 ml or 500 ml; in some embodiments, 150 to 240 or 250 ml liquid.

In some embodiments, the irrigation is with from or from about 100 to or to about 500 ml liquid. In some embodiments, the irrigation is with from or from about 100 to or to about 450, from or from about 100 to or to about 400, from or from about 100 to or to about 350, from or from about 100 to or to about 300, from or from about 100 to or to about 250, from or from about 100 to or to about 200, from or from about 100 to or to about 150, from or from about 150 to or to about 500, from or from about 150 to or to about 450, from or from about 150 to or to about 400, from or from about 150 to or to about 350, from or from about 150 to or to about 300, from or from about 150 to or to about 250, from or from about 150 to or to about 200, from or from about 200 to or to about 500, from or from about 200 to or to about 450, from or from about 200 to or to about 400, from or from about 200 to or to about 350, from or from about 200 to or to about 300, from or from about 200 to or to about 250, from or from about 250 to or to about 500, from or from about 250 to or to about 450, from or from about 250 to or to about 400, from or from about 250 to or to about 350, from or from about 250 to or to about 300, from or from about 300 to or to about 500, from or from about 300 to or to about 450, from or from about 300 to or to about 400, from or from about 300 to or to about 350, from or from about 350 to or to about 500, from or from about 350 to or to about 450, from or from about 350 to or to about 400, from or from about 400 to or to about 500, from or from about 400 to or to about 450, or from or from about 450 to or to about 500 ml liquid.

In some embodiments, the liquid is water or saline.

In some embodiments, administration via nasal irrigation is carried out daily.

In some embodiments, administration via nasal irrigation is carried out every 1, 2, 3, 4, 5, 6, or 7 days.

Thus, provided herein is a kit comprising a P-gp inhibitor, e.g., verapamil, a corticosteroid, e.g., mometasone, and a device suitable for high volume, low positive pressure nasal irrigation. In some embodiments, the kit further comprises sodium chloride. In some embodiments, the device is selected from the group consisting of a spray bottle, a pump, a squirt bottle, a nebulizer, a gravity-based pressure using a vessel with a nasal spout, or an exhalation delivery system.

In some embodiments, the P-gp inhibitor, e.g., verapamil, and the corticosteroid, e.g., mometasone, is administered locally to the subject's nasal passage and sinuses by nasal spray, nebulization, or nasal drop.

Thus, also provided herein are kits comprising a P-gp inhibitor, e.g., verapamil, a corticosteroid, e.g., mometasone, and a nasal spray bottle, nebulization device, and/or nasal drops.

In some embodiments, the P-gp inhibitor, e.g., verapamil, and/or the corticosteroid, e.g., mometasone, are administered by medical implant, e.g., as part of a drug eluting stent.

As used herein, the term “medical implant” refers to a device that is placed into a surgically or naturally formed cavity of a body, e.g., a human body. In some embodiments, the device is intended to remain in the cavity for a period of 30 days or more.

As used herein, the term “drug eluting stent” refers to a mesh tube that emits a drug over time. The amount of therapeutic agent in a drug eluting stent is characterized by the amount of drug per surface area of the tube.

In some embodiments, the implantable matrix is a polymeric matrix, e.g., a bioresorbable polymeric matrix. See, e.g., Wu et al., “In-Office Corticosteroid Placement in the Management of Chronic Rhinosinusitis,” Ear, Nose & Throat Journal 2020:doi.org/10.1177/0145561320982193; Lelegren et al., “Intraoperative Applications of Topical Corticosteroid Therapy for Chronic Rhinosinusitis,” Ear, Nose & Throat Journal 2020:doi.org/10.1177/0145561320970100.

Suitable bioresorbable polymeric matrices are described, e.g., in Douglas et al., “Phase 1 Clinical Study to Assess the Safety of a Novel Drug Delivery System Providing Long-Term Topical Steroid Therapy for Chronic Rhinosinusitis,” International Forum of Allergy & Rhinology 9(4):doi.org/10.1002/alr.22288 (2019), WO2010135433A1, WO2013052739A1, WO2013158619A2, WO2014126957A1, WO2014172319A1, WO2017004268A1, U.S. Pat. No. 10,219,894B2, US20160374800A1, U.S. Pat. No. 10,232,082B2, US10278812B2, W02018195484A1, US20200368388A1, U.S. Pat. No. 10,806,568B2, U.S. Pat. No. 10,864,298B2, US20200316253A1, and US20210068945A1,

Thus, described herein are medical implants comprising a P-gp inhibitor, e.g., verapamil, and/or a corticosteroid, e.g., mometasone. In some embodiments, the medical implant comprises both a P-gp inhibitor, e.g., verapamil, and a corticosteroid, e.g., mometasone.

In some embodiments, the implant comprises polylactice-co-glycolide, synthetic polyurethane foam, carboxymethyl-cellulose, hyaluronic acid, calcium alginate, gelatin, hydroxylated polyvinyl acetate, hydroxylated polyvinyl acetate, fibrinogen,

In some embodiments, the polymeric matrix is a scaffold. In some embodiments, the scaffold comprises i) a first layer comprising the P-gp inhibitor, e.g., verapamil, and/or the corticosteroid, e.g., mometasone and a biodegradable polymer matrix and ii) a therapeutic-agent-free polymer topcoat layer positioned on the first layer.

In some embodiments, the implant delivers the P-gp inhibitor, e.g., verapamil, and/or corticosteroid, e.g., mometasone, e.g., mometasone furoate, from the middle meatus to a sinus cavity of a patient, e.g., a human patient. In some embodiments, the sinus cavity is selected from the group consisting of a maxillary sinus cavity, a frontal sinus cavity, a sphenoid sinus cavity, an ethmoid sinus cavity, and combinations thereof.

In some embodiments, the P-gp inhibitor, e.g., verapamil, is administered by the same method as the corticosteroid, e.g., mometasone, and optionally e.g., by any of the methods described herein. In some embodiments, the P-gp inhibitor and corticosteroid are administered at the same time and by the same method. In some embodiments, the P-gp inhibitor and corticosteroid are administered sequentially by the same method. In embodiments where the method comprises administering an antibiotic, the antibiotic may also be administered by the same method, e.g., at the same time and by the same method or sequentially by the same method.

In some embodiments, the P-gp inhibitor, e.g., verapamil, is administered by a different method than the corticosteroid, e.g., mometasone, e.g., by any of the methods described herein for administration of a P-gp inhibitor and corticosteroid. That is, where administration is contemplated with respect to the combination of P-gp inhibitor, e.g., verapamil, and corticosteroid, e.g., mometasone, the same administration method can be used for the P-gp inhibitor, e.g., verapamil, or the corticosteroid, e.g., mometasone, alone. In some embodiments, the P-gp inhibitor and corticosteroid are administered at the same time using different methods. In some embodiments, the P-gp inhibitor and corticosteroid are administered sequentially using different methods. In embodiments where the method comprises administering an antibiotic, the antibiotic may also be administered by a different method than the P-gp inhibitor and/or corticosteroid, e.g., a different method than both the P-gp inhibitor and the corticosteroid or a different method than the P-gp inhibitor but the same method as the corticosteroid or a different method than the corticosteroid but the same method as the antibiotic. In any of these embodiments, the antibiotic may be administered at the same time as the P-gp inhibitor and/or the corticosteroid or sequentially with the P-gp inhibitor and/or corticosteroid.

In some embodiments, a kit for treating rhinosinusitis in a subject is provided. Such a kit can comprise a pharmaceutical composition comprising or consisting of an effective amount of a P-gp inhibitor, e.g., verapamil, and a corticosteroid, e.g., mometasone, optionally an antibiotic, and optionally a device for delivering the pharmaceutical composition to the subject's nasal passage and sinuses, such as a squeeze bottle. The P-gp inhibitor and corticosteroid (and optional antibiotic) can be provided in a concentrated form, and the kit can also include sufficient salts to provide an isotonic (normal saline) solution and/or a hypertonic saline solution for comfortable nasal irrigation upon addition of water (e.g., distilled or other clean water, not necessarily sterile). In some embodiments, the salts comprise sodium chloride and a buffering agent, e.g., sodium bicarbonate, e.g., sufficient sodium chloride to provide a final concentration of 0.8-1%, e.g., 0.9 percent sodium chloride, and buffering agent to provide a pH of 4.5 to 7.

Each dose of the the P-gp inhibitor and corticosteroid (and optional antibiotic) and salt can be provided in a single container or in multiple individual containers. The containers can be, e.g., a bottle, vial, ampoule, packet or sachet.

The kit can also include one or more viscosity enhancing agents, such as a cellulose polymer or polyethylene glycol (PEG); preservatives; and/or surfactants, which can be incorporated into, e.g. mixed in with, one or more of the P-gp inhibitor and corticosteroid (and optional antibiotic) and salt. See, e.g., US20180104253.

In addition, the kit can include a bottle, e.g., a reusable bottle, e.g., as known in the art (see also U.S. Pat. Nos. 1,603,758; 1,856,811; 3,847,145; 5,649,530; 6,328,718; 6,520,284; 6,736,792; 6,907,879; 8,162,921; US PGPUB 2006/0276743; 2009/0202665; 2008/0221507; WO 2006/051206; WO 2008/058160; US2017/0128659, U.S. Pat. Nos. 6,520,374, 6,669,059, and 9,623,170B inter alia).

### Dosing

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The following examples are included for illustrative purposes only and are not intended to limit the scope of the invention.

### Example 1: P-glycoprotein Inhibition with Verapamil Overcomes Mometasone Resistance in Chronic Sinusitis with Nasal Polyps

P-glycoprotein (P-gp) is a membrane efflux pump that is overexpressed in Chronic Rhinosinusitis with Nasal Polyps (CRSwNP) and promotes type 2 inflammation. Some, but not all, glucocorticoids (GC) are substrates of P-gp; thus, overexpression of P-gp may additionally contribute to GC resistance in CRSwNP. The present study determine whether P-gp inhibition using verapamil enhances mometasone retention and efficacy in nasal polyp explants.

In the present IRB approved study, organotypic polyp explants were exposed to mometasone (4.15 μg/mL) and verapamil (125 μg/mL) as mono or combination therapy. The effect of verapamil on mometasone intracellular retention over time was determined using HPLC. The effect of verapamil on the anti-inflammatory effect of mometasone was determined using ELISA for secreted IL-5, IL-6, and IL-17. Groups were compared using (unpaired t-test).

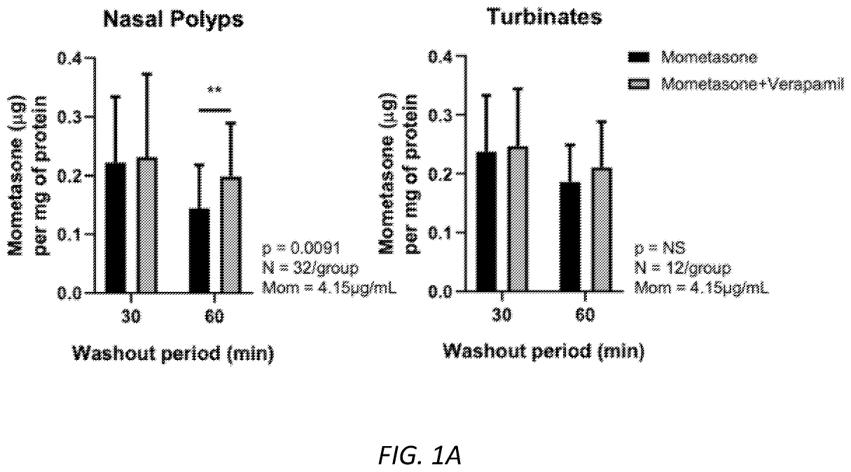

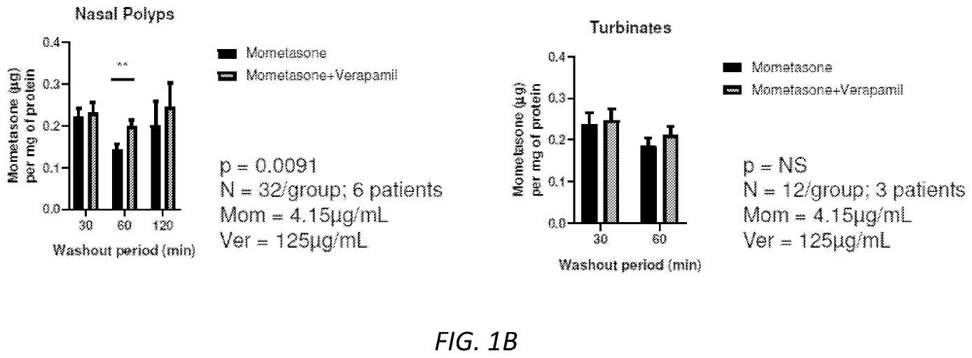

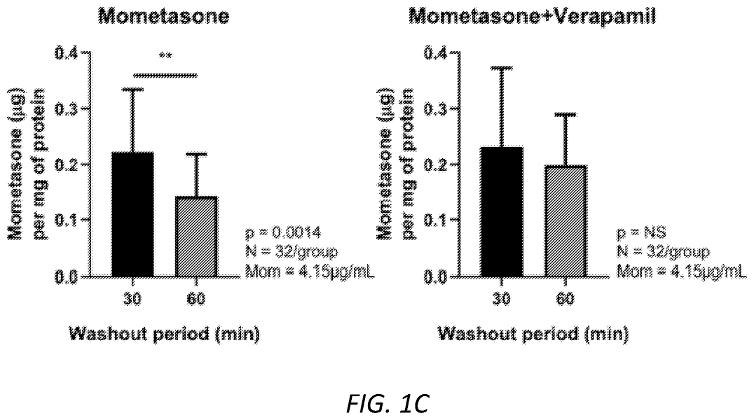

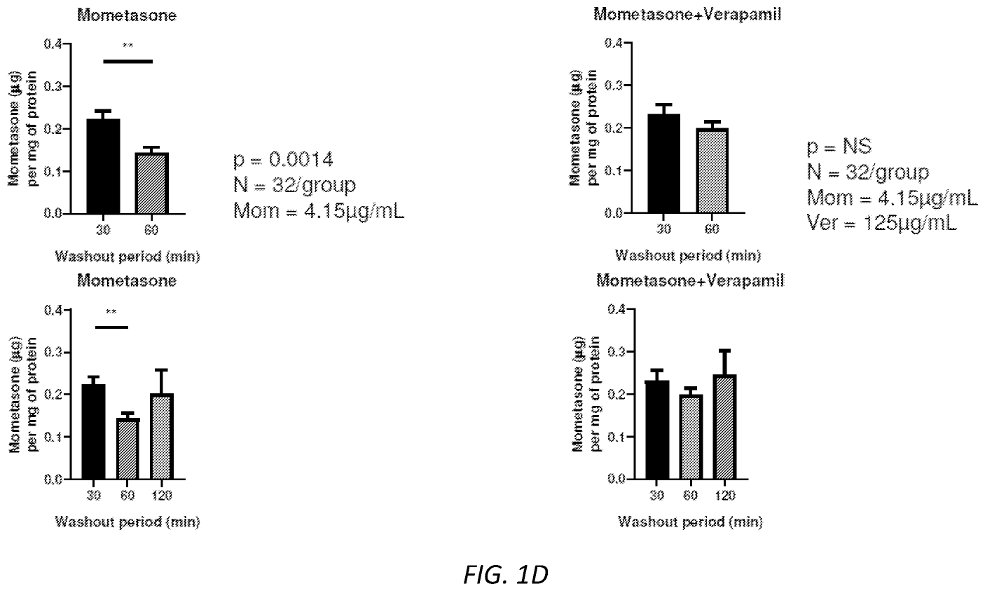

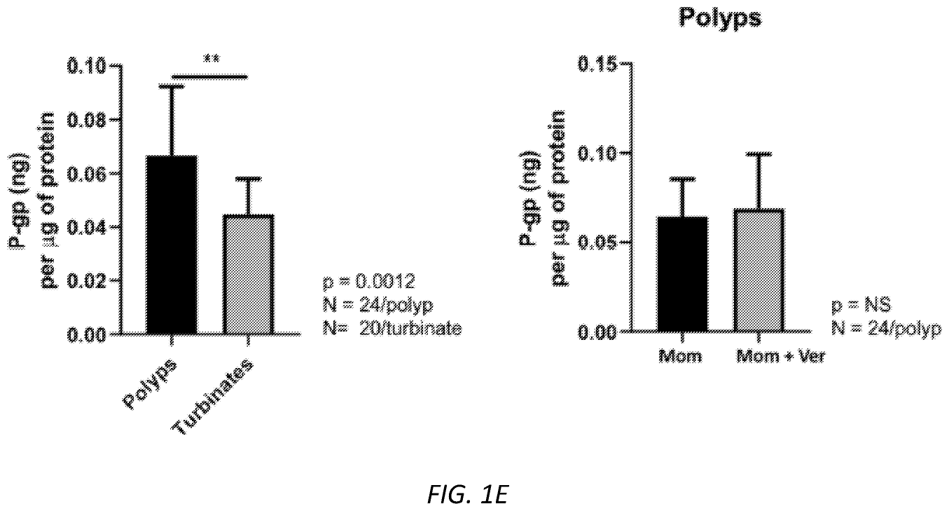

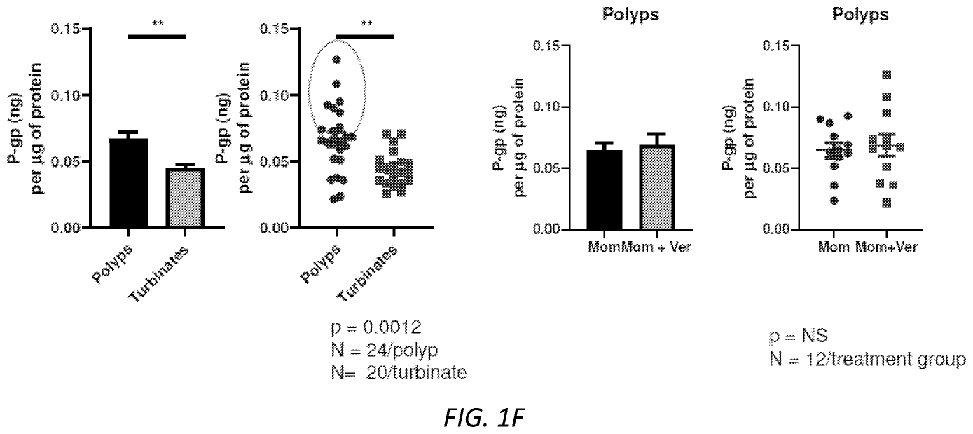

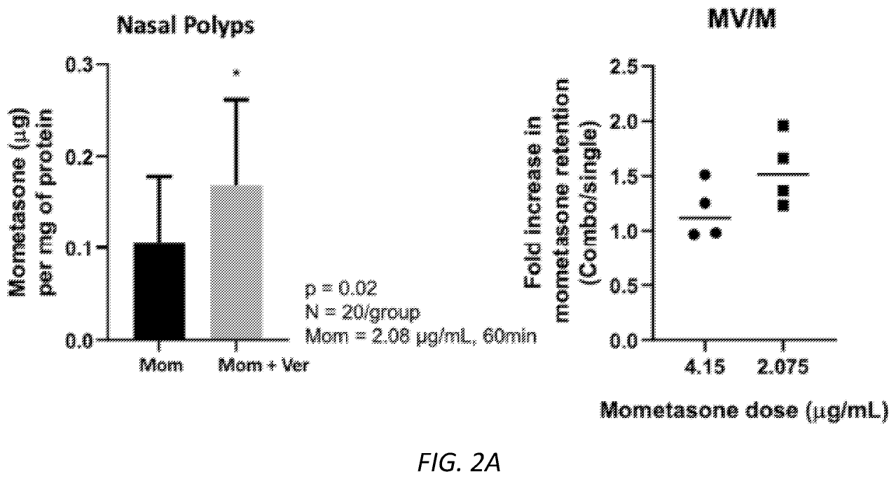

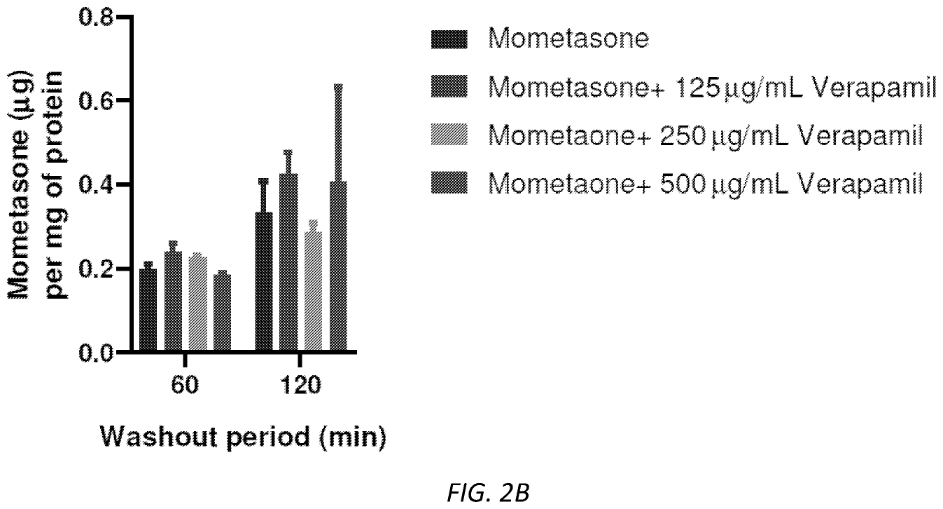

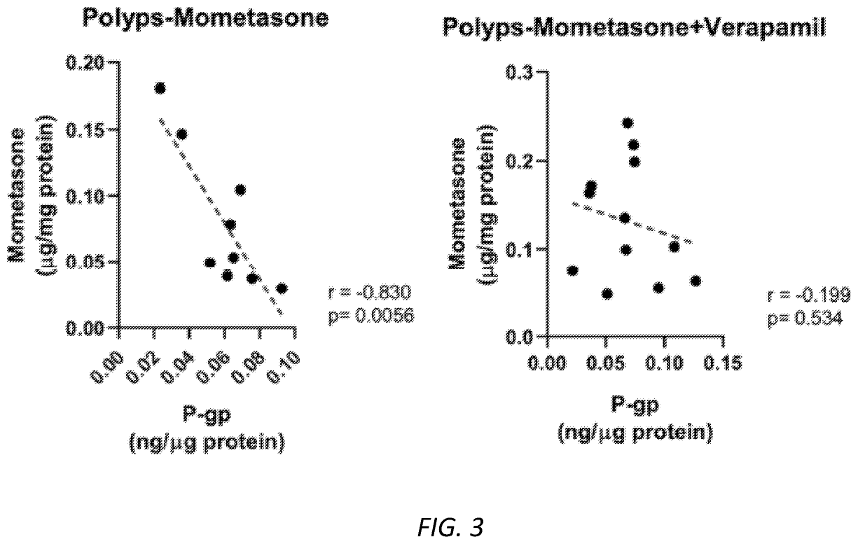

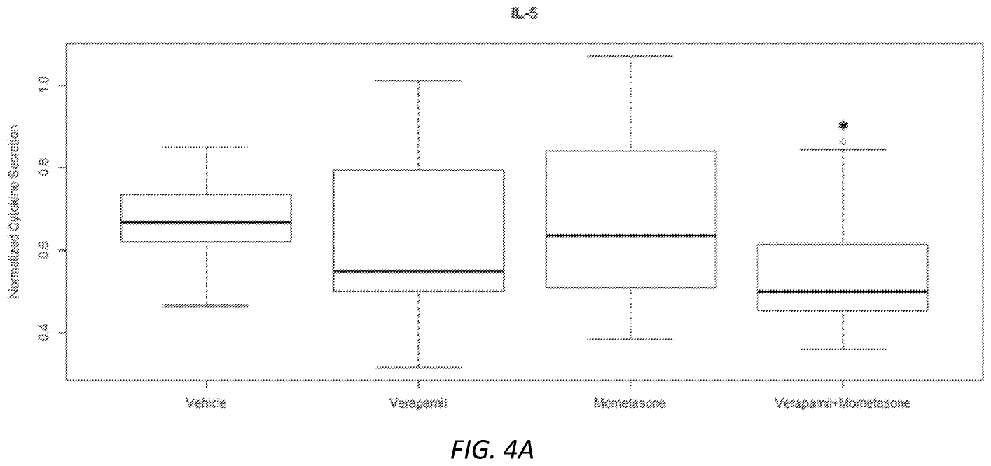

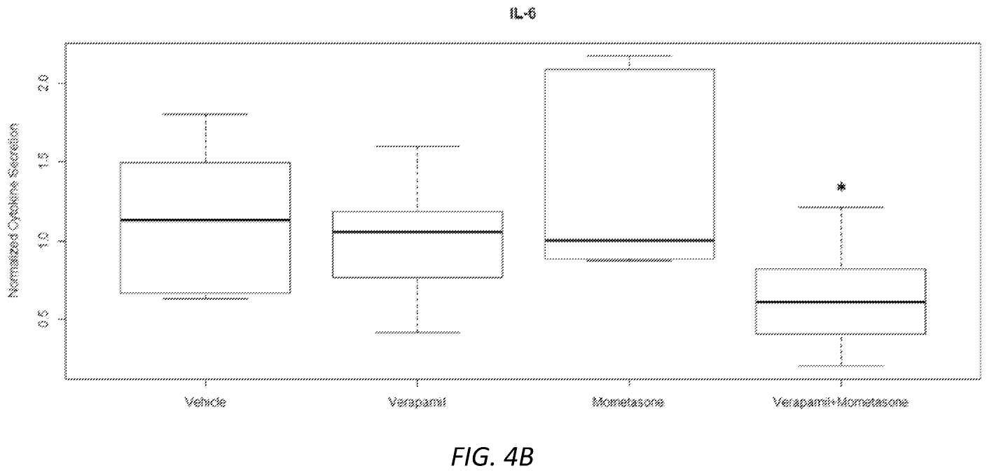

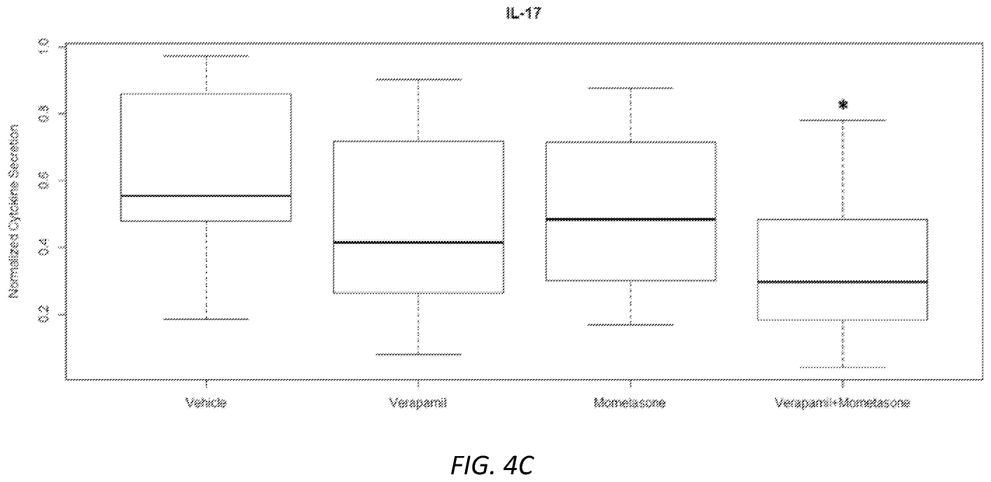

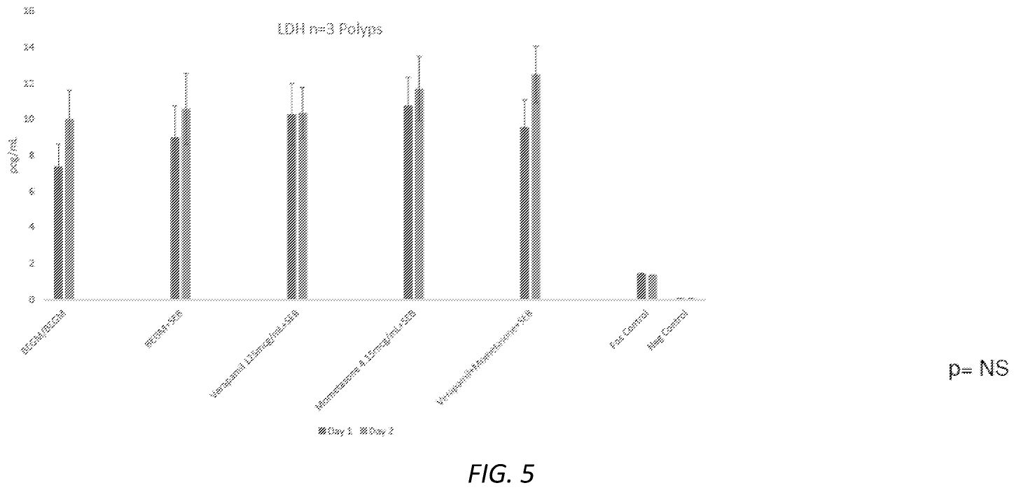

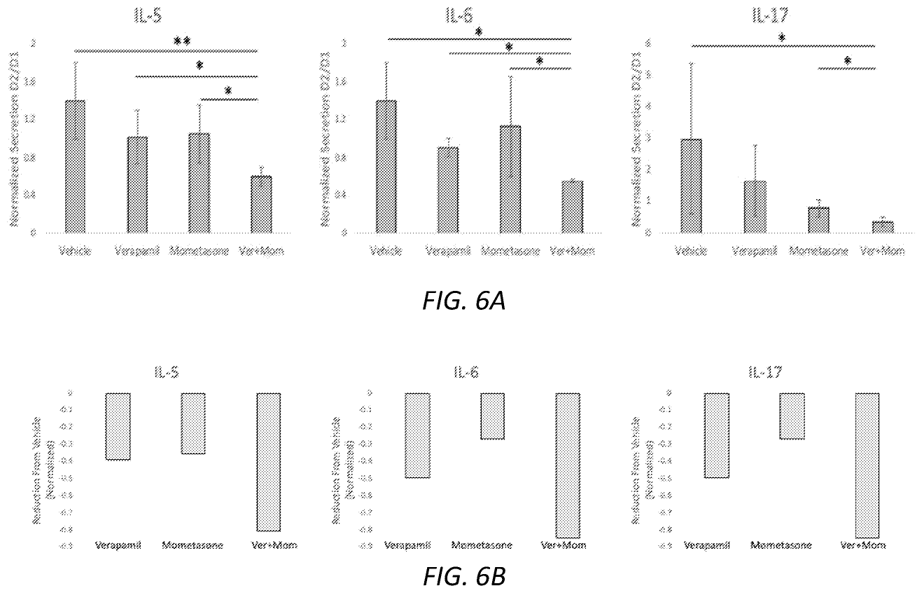

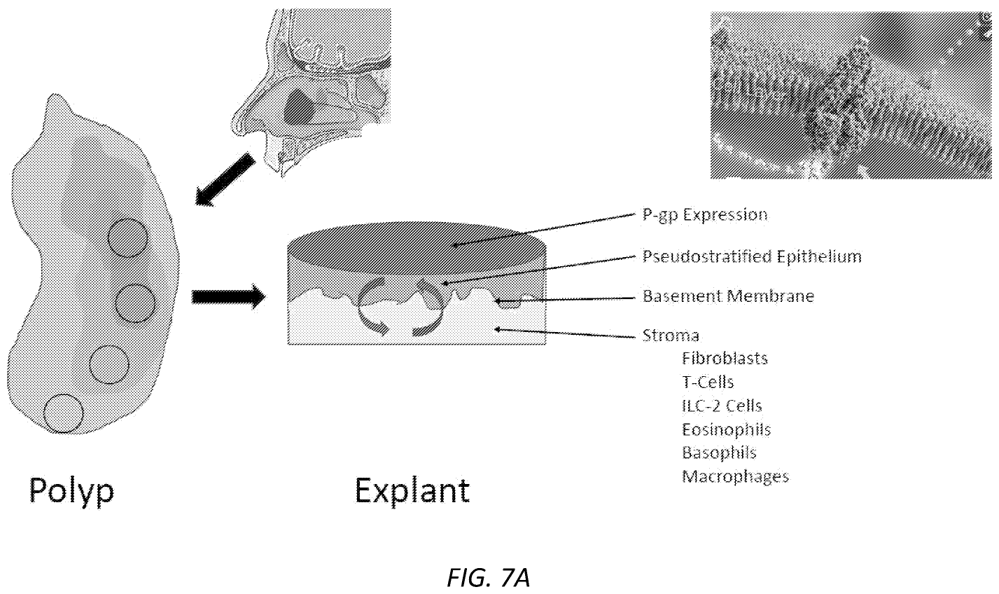

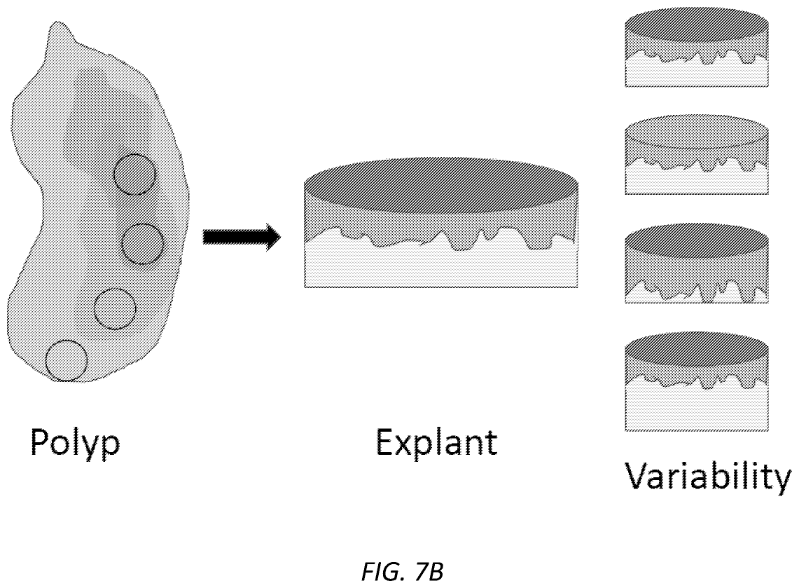

As shown in FIGS. 1-7, P-gp expression strongly and significantly inversely correlated with mometasone retention lhr after exposure (r=−0.83, p<0.01). P-gp inhibition reversed this effect and significantly improved mometasone retention at 1 hour relative to mometasone alone (p<0.01). The combination of mometasone and verapamil significantly reduced IL-5, IL-6, and IL-17 secretion relative to vehicle control (p=0.01, p<0.001, and p=0.01) and outperformed either treatment alone.

This study confirmed that mometasone is a substrate of P-gp, exhibiting a nearly 6-fold reduction in intracellular retention between the lowest and highest P-gp expressing polyp explants. This P-gp mediated resistance was successfully reversed by the addition of the P-gp inhibitor verapamil. Verapamil further significantly enhanced the anti-inflammatory effect of mometasone when given as a combination therapy. This is the first data to show that: 1) Mometasone is a substrate for P-gp; 2) P-gp overexpression in Nasal polyps results in a significant reduction in intracellular retention of mometasone; 3) The addition of Verapamil significantly increased intracellular mometasone retention in Nasal polyps; and 4) The combination of mometasone and verapamil has superior anti-inflammatory effects than either drug alone.

### Example 2: P-glycoprotein Inhibition with Verapamil Overcomes Mometasone Resistance in Chronic Sinusitis with Nasal Polyps

### Materials and Methods

Materials

Mometasone furoate, Verapamil hydrochloride, dexamethasone-21-acetate and CelLytic™ MT cell lysis reagents were purchased from Sigma Aldrich (St. Louis, Mo.). Bronchial epithelial growth medium (BEGM) was purchased from Lonza (Basel, Switzerland). Pierce™ BCA Protein Assay Kit was purchased from ThermoFisher Scientific (Waltham, Mass.). Human P-gp ELISA kit was purchased from Cedarlane (Burlington, N.C.). Custom Human Cytokine Q-Plex Array was purchased from Quansys Biosciences (Logan, Utah). CytoTox 96® Non-Radioactive Cytotoxicity Assay was purchased from Promega (Madison, Wis.). All solvents used were purchased from Fisher scientific and were HPLC grade.

Primary Human Mucosal Sampling

Tissue sampling was approved by the Massachusetts Eye and Ear Institutional Review Board. All samples were taken from patients who had not been exposed to antibiotics or steroids for at least 4 weeks. Inclusion criteria included patients diagnosed with CRSwNP and healthy patients (i.e. controls) undergoing turbinate reduction surgery for non-inflammatory disease. Exclusion criteria included patients with ciliary dysfunction, autoimmune disease, cystic fibrosis, immunodeficiency and smoking. Among controls, additional exclusion criteria included the presence of allergy or asthma. Diagnostic criteria for asthma, aspirin-exacerbated respiratory disease (AERD), and allergic rhinitis were based on both clinical history and allergy testing. Not all patients were used in all experiments but rather within each results subsection the patients used were the same between control and experimental groups to maintain consistency.

Explant Incubation for Mometasone Retention Evaluation

Harvested polyps; from patients washed out of oral or topical steroid for 4 weeks, were immediately sectioned into 5-mm explants using standard biopsy punch (Integra™ Meltex™ ), taking care to maintain an intact epithelial layer in each explant as previously described (13). Explants were individually placed in tubes containing 350 μL of hydrocortisone-free BEGM containing 0.5 μg/mL of Staphylococcus aureus enterotoxin B (SEB) and either mometasone alone or a combination of mometasone and the P-gp inhibitor, verapamil (2.08, 4.15 μg/mL and 125 μg/mL for mometasone and verapamil concentrations, respectively). Tubes were incubated at 37° C., 5% CO2 for 30 minutes, media was then removed, and explants washed with BEGM to remove any surface-bound drug. Explants were then incubated in mometasone-free BEGM for 30, 60 or 120 minutes as washout periods. For the combination group, explants were washed out in BEGM containing 125 μg/mL verapamil to maintain the P-gp blockade. At the end of the washout period, explants were rinsed with BEGM and stored at −80° C. for later analysis of mometasone concentration. Turbinate (control) tissues were sectioned and treated similar to polyps, with exception that the BEGM used for incubation did not contain SEB.

Quantification of Mometasone Retention and P-gp levels in Explants

Explants were homogenized in 400 μL of cell lytic buffer and the homogenates (200 μL) were spiked with 1 μL of dexamethasone-21-acetate ethanolic stock, used as an internal standard for extraction. The spiked homogenates were extracted with 800 μL of ethyl acetate by vortexing for 15 min. The organic layer was separated by centrifugation at 4000 x g for 10 min at 4° C., and 600 μL of it was transferred to new tubes and dried in air. Dried films were reconstituted in 75 μL of acetonitrile and analyzed with high performance liquid chromatography (HPLC) for mometasone concentration. Standards for HPLC analysis were prepared by spiking tissue homogenate (125 mg/mL in cell lytic buffer, from same patient's tissue) with mometasone (from standard stocks) to final concentration of 5, 2.5, 1.25, 0.63, 0.13, 0.08 and 0.04 μg/mL. Standards were treated similarly for extraction and analysis. The remainder of the tissue homogenates were centrifuged at 13000 x g for 20 min at 4° C. and supernatants were collected for protein quantification using a BCA assay and for analysis of P-gp expression using an ELISA kit following the manufacturer's protocol.

Explant Incubation for Anti-IL-5 Effect Evaluation

Polyps (sectioned as above) were placed in tubes containing 350 μL of hydrocortisone-free BEGM containing 0.5 μg/mL of SEB and were incubated at 37° C., 5% CO2. After 24 h, media was collected and stored at −80° C. (Day 1) and replaced with 350 μL of BEGM containing 0.5 μg/mL of SEB and either mometasone or a combination of mometasone and verapamil (4.15 μg/mL and 125 μg/mL for mometasone and verapamil concentrations, respectively). After 24 h in the treatment condition, media was collected and stored at −80° C. (Day 2) for later cytokine and cytotoxicity analysis. Explants incubated in media (BEGM or BEGM containing 0.5 μg/mL of SEB) with no treatment were included as controls. Secreted IL-5 was quantified in day 1 and 2 samples, using the Quansys Q-Plex array. Cytokine secretion in response to the different treatments was normalized to Day 1 secretion level and expressed as Day2/Day1 ratio to make direct comparisons between polyp samples as previously described (14). The released LDH in the samples was assayed using CytoTox 96® cytotoxicity assay, as an indicator for cytotoxicity caused by the treatment groups.

Statistical Analysis

All data is presented as means±standard error of the mean (SEM). Statistical analyses were performed with GraphPad Prism 8 (La Jolla, Calif., USA). Values falling outside 1.5 times the interquartile range of their respective data set were considered outliers and indiscriminately excluded from analysis as previously described (13). Data were analyzed by Shapiro-Wilk test for normality, two-tailed student t-test, two-way ANOVA, Mann-Whitney test, Kruskal-Wallis test or Pearson correlation, as indicated. A p-value of <0.05 was considered statistically significant.

### Results

P-gp Expression Inversely Correlated with Mometasone Retention

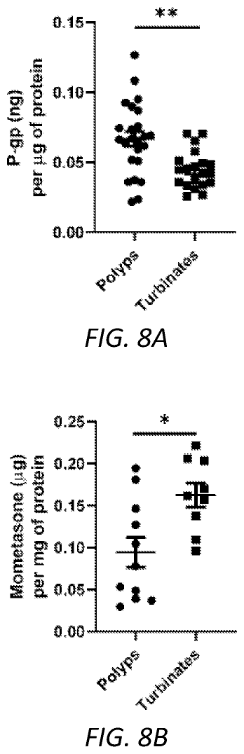

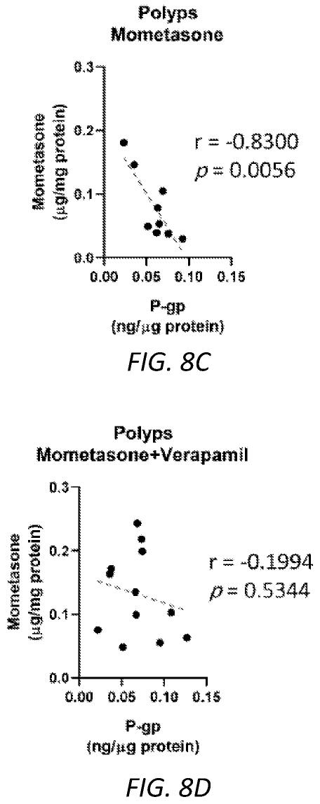

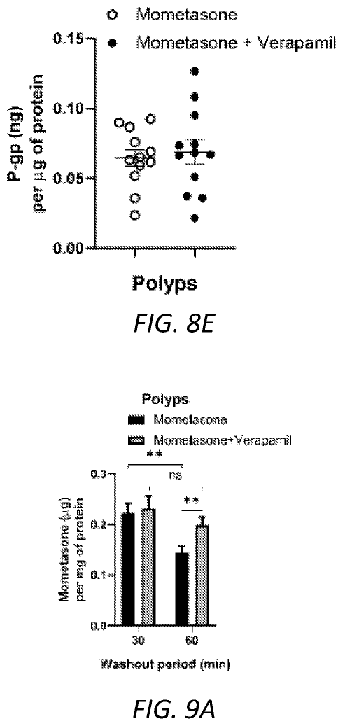

P-gp concentrations in the polyp explants were significantly higher than in control turbinate tissues (p<0.01, unpaired two-tailed t-test) (FIG. 8A). This P-gp overexpression in polyps resulted in significantly lower tissue mometasone retention than in the lower P-gp expressing turbinates (p<0.05, unpaired two-tailed t-test) (FIG. 8B). P-gp expression in polyp explants correlated significantly and inversely with mometasone retention (Pearson's r=−0.8300, p=0.0056). Polyps with high P-gp expression levels retained the least mometasone after a 60 min washout period (FIG. 8C), with a 6-fold reduction in retention between the lowest and highest P-gp expressing polyp explants. This strong and significant inverse correlation was abrogated (Pearson's r=−0.1994, p=0.5344) when mometasone was treated in combination with the P-gp inhibitor, verapamil (FIG. 8D). There was no significant difference between P-gp levels in polyps exposed to either mometasone alone or in combination with verapamil (FIG. 8E).

Verapamil Enhanced Mometasone Retention in Organotypic Polyp Explants in a Dose Dependent Manner

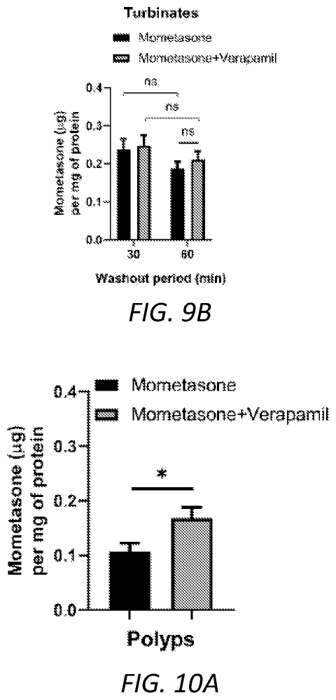

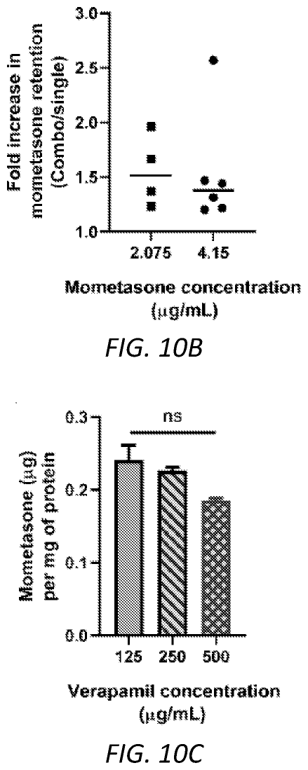

P-gp inhibition with verapamil did not influence the initial mometasone uptake in polyp explants, as indicated by mometasone tissue concentration after 30 min of washout. However, it resulted in significantly improved retention of mometasone in polyps over time (FIG. 9A). Polyps exposed to mometasone only demonstrated mometasone efflux over 1 hour (30 min versus 60 min washout, p<0.01, unpaired two-tailed t-test). In contrast, co-treatment with verapamil maintained mometasone tissue concentration over the entire 60 min washout period and resulted in significantly higher tissue mometasone concentration as compared to the mometasone-only group (154%±52%, p<0.01, unpaired two-tailed t-test). Of note, verapamil did not influence mometasone retention in the control tissue (FIG. 9B) at any of the washout time points. It is noteworthy that the significant enhancement in mometasone retention upon verapamil co-treatment was also observed at half the mometasone dose (156%±32%, p<0.05,Mann-Whitney test) (FIG. 10A), resulting in a similar fold increase in mometasone level from the single treatment (FIG. 10B). Increasing verapamil concentrations did not result in a concentration-dependent response (FIG. 10C).

Verapamil Significantly Enhanced the Anti-IL-5 Effect of Mometasone

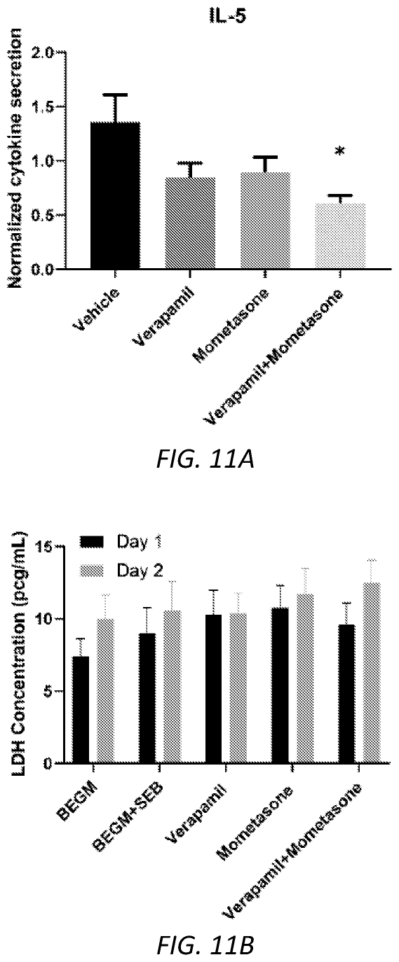

We next tested the influence of P-gp inhibition on the anti-inflammatory effect of mometasone. Co-treatment of verapamil with mometasone for 24 h significantly decreased the secretion of IL-5 as compared to both the untreated explants and those treated with either monotherapy alone (p<0.05, Kruskal-Wallis test) (FIG. 11A), without inducing cytotoxicity (FIG. 11B).

### REFERENCES

1. Rosenfeld R M, Piccirillo J F, Chandrasekhar S S, Brook I, Ashok Kumar K, Kramper M, et al. Clinical practice guideline (update): adult sinusitis. Otolaryngol Head Neck Surg. 2015; 152(2 Suppl):S1 -S39.

2. Fokkens W J, Lund V J, Mullol J, Bachert C, Alobid I, Baroody F, et al. European Position Paper on Rhinosinusitis and Nasal Polyps 2012. Rhinol. 2012; 23:1-298.

3. Scadding G K, Durham S R, Mirakian R, Jones N S, Drake-Lee A B, Ryan D, et al. BSACI guidelines for the management of rhinosinusitis and nasal polyposis. Clin Exp Allergy. 2008; 38(2):260-75.

4. Desrosiers M, Evans G A, Keith P K, Wright E D, Kaplan A, Bouchard J, et al. Canadian clinical practice guidelines for acute and chronic rhinosinusitis. J Otolaryngol Head Neck Surg. 2011; 40 Suppl 2:S99-193.

5. Derendorf H, Meltzer E O. Molecular and clinical pharmacology of intranasal corticosteroids: clinical and therapeutic implications. Allergy. 2008; 63(10):1292-300.

6. Vaidyanathan S, Barnes M, Williamson P, Hopkinson P, Donnan P T, Lipworth B. Treatment of chronic rhinosinusitis with nasal polyposis with oral steroids followed by topical steroids: a randomized trial. Ann Intern Med. 2011; 154(5):293-302.

7. Rupa V, Jacob M, Mathews M S, Seshadri M S. A prospective, randomised, placebo-controlled trial of postoperative oral steroid in allergic fungal sinusitis. Eur Arch Otorhinolaryngol. 2010; 267(2):233-8.

8. Van Zele T, Gevaert P, Holtappels G, Beule A, Wormald P J, Mayr S, et al. Oral steroids and doxycycline: two different approaches to treat nasal polyps. J Allergy Clin Immunol. 2010; 125(5):1069-76 e4.

9. Hissaria P, Smith W, Wormald P J, Taylor J, Vadas M, Gillis D, et al. Short course of systemic corticosteroids in sinonasal polyposis: a double-blind, randomized, placebo-controlled trial with evaluation of outcome measures. J Allergy Clin Immunol. 2006; 118(1):128-33.

10. Gurrola J, 2nd, Borish L. Chronic rhinosinusitis: Endotypes, biomarkers, and treatment response. J Allergy Clin Immunol. 2017; 140(6):1499-508.

11. Bleier B S. Regional expression of epithelial MDR1/P-glycoprotein in chronic rhinosinusitis with and without nasal polyposis. Int Forum Allergy Rhinol. 2012; 2(2):122-5.

12. Feldman R E, Lam A C, Sadow P M, Bleier B S. P-glycoprotein is a marker of tissue eosinophilia and radiographic inflammation in chronic rhinosinusitis without nasal polyps. Int Forum Allergy Rhinol. 2013; 3(8):684-7.

13. Bleier B S, Singleton A, Nocera A L, Kocharyan A, Petkova V, Han X. P-glycoprotein regulates Staphylococcus aureus enterotoxin B-stimulated interleukin-5 and thymic stromal lymphopoietin secretion in organotypic mucosal explants. Int Forum Allergy Rhinol. 2016; 6(2):169-77.

14. Bleier B S, Kocharyan A, Singleton A, Han X. Verapamil modulates interleukin-5 and interleukin-6 secretion in organotypic human sinonasal polyp explants. Int Forum Allergy Rhinol. 2015; 5(1):10-3.

15. Bleier B S, Nocera A L, Iqbal H, Hoang J D, Alvarez U, Feldman R E, et al. P-glycoprotein promotes epithelial T helper 2-associated cytokine secretion in chronic sinusitis with nasal polyps. Int Forum Allergy Rhinol. 2014; 4(6):488-94.

16. Kocharyan A, Feldman R, Singleton A, Han X, Bleier B S. P-glycoprotein inhibition promotes prednisone retention in human sinonasal polyp explants. Int Forum Allergy Rhinol. 2014; 4(9):734-8.

17. Farrell R J, Murphy A, Long A, Donnelly S, Cherikuri A, O'Toole D, et al. High multidrug resistance (P-glycoprotein 170) expression in inflammatory bowel disease patients who fail medical therapy. Gastroenterology. 2000; 118(2):279-88.

18. Khakzad M R, Mirsadraee M, Mohammadpour A, Ghafarzadegan K, Hadi R, Saghari M, et al. Effect of verapamil on bronchial goblet cells of asthma: an experimental study on sensitized animals. Pulm Pharmacol Ther. 2012; 25(2):163-8.

19. Tsuruo T, Iida H, Yamashiro M, Tsukagoshi S, Sakurai Y. Enhancement of vincristine- and adriamycin-induced cytotoxicity by verapamil in P388 leukemia and its sublines resistant to vincristine and adriamycin. Biochem Pharmacol. 1982; 31(19):3138-40.

20. Hashioka S, Klegeris A, McGeer P L. Inhibition of human astrocyte and microglia neurotoxicity by calcium channel blockers. Neuropharmacology. 2012; 63(4):685-91.

21. Li G, Qi X P, Wu X Y, Liu F K, Xu Z, Chen C, et al. Verapamil modulates LPS-induced cytokine production via inhibition of NF-kappa B activation in the liver. Inflamm Res. 2006; 55(3):108-13.

22. Matsumori A, Nishio R, Nose Y. Calcium channel blockers differentially modulate cytokine production by peripheral blood mononuclear cells. Circ J. 2010; 74(3):567-71.

23. Miyake M M, Nocera A, Levesque P, Guo R, Finn C A, Goldfarb J, et al. Double-blind placebo-controlled randomized clinical trial of verapamil for chronic rhinosinusitis with nasal polyps. J Allergy Clin Immunol. 2017; 140(1):271-3.

24. Forwith K D, Chandra R K, Yun P T, Miller S K, Jampel H D. ADVANCE: a multisite trial of bioabsorbable steroid-eluting sinus implants. Laryngoscope. 2011; 121(11):2473-80.

25. Douglas R G, Psaltis A J, Rimmer J, Kuruvilla T, Cervin A, Kuang Y. Phase 1 clinical study to assess the safety of a novel drug delivery system providing long-term topical steroid therapy for chronic rhinosinusitis. Int Forum Allergy Rhinol. 2019; 9(4):378-87.

26. Sindwani R, Han J K, Soteres D F, Messina J C, Carothers J L, Mahmoud R A, et al. NAVIGATE I: Randomized, Placebo-Controlled, Double-Blind Trial of the Exhalation Delivery System With Fluticasone for Chronic Rhinosinusitis With Nasal Polyps. Am J Rhinol Allergy. 2019; 33(1):69-82.

27. Szefler S J. Pharmacokinetics of intranasal corticosteroids. J Allergy Clin Immunol. 2001; 108(1 Suppl):S26-31.

28. Mares-Samano S, Badhan R, Penny J. Identification of putative steroid-binding sites in human ABCB1 and ABCG2. Eur J Med Chem. 2009; 44(9):3601-11.

29. Webster J I, Carlstedt-Duke J. Involvement of multidrug resistance proteins (MDR) in the modulation of glucocorticoid response. J Steroid Biochem Mol Biol. 2002; 82(4-5):277-88.

30. Bleier B S, Nocera A L, Iqbal H, Hoang J D, Feldman R E, Han X. P-glycoprotein functions as an immunomodulator in healthy human primary nasal epithelial cells. Int Forum Allergy Rhinol. 2013; 3(6):433-8.

31. Nocera A L, Meurer A T, Miyake M M, Sadow P M, Han X, Bleier B S. Secreted P-glycoprotein is a noninvasive biomarker of chronic rhinosinusitis. Laryngoscope. 2017; 127(1):E1-E4.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating rhinosinusitis in a subject, the method comprising:
identifying a subject having rhinosinusitis; and
administering to the subject an effective amount of verapamil and mometasone.

2. A method of enhancing corticosteroid retention in a subject's sinonasal epithelial cells, the method comprising:
identifying a subject overexpressing P-gp in the subject's sinonasal epithelial cells; and
administering to the subject an effective amount of verapamil and mometasone.

3. A method of reducing inflammation in a subject's sinonasal epithelial cells, the method comprising:
identifying a subject overexpressing P-gp in the subject's sinonasal epithelial cells; and
administering to the subject an effective amount of verapamil and mometasone.

4. The method of claim 2 or claim 3, wherein identifying a subject overexpressing P-gp in the subject's sinonasal epithelial cells comprises providing a sample comprising nasal secretions, preferably comprising nasal mucus, from a subject; determining a level of soluble p-glycoprotein (P-gp) in the sample; and comparing the level of P-gp in the sample to a reference level of P-gp; wherein a level of P-gp in the sample above the reference level indicates that the subject overexpresses P-gp.

5. The method of any of the preceding claims, wherein the subject has chronic rhinosinusitis (CRS) or CRS with nasal polyps (CRSwNP).

6. The method of any of the preceding claims, wherein the verapamil and mometasone are administered systemically.

7. The method of any of the preceding claims, wherein the verapamil and mometasone are administered locally to the subject's nasal passage and sinuses.

8. The method of claim 7, wherein the verapamil and mometasone are delivered to the subject's nasal passage and sinuses by nasal irrigation.

9. The method of claim 8, wherein the nasal irrigation is high volume, low positive pressure nasal irrigation.

10. The method of claim 9, wherein the verapamil and mometasone are delivered to the subject's nasal passage and sinuses by high volume, low positive pressure nasal irrigation with a saline solution.

11. The method of claim 10, wherein the saline solution is an isotonic saline solution.

12. The method of claim 10, wherein the saline solution is a hypertonic saline solution.

13. The method of claim 12, wherein the hypertonic saline solution is about a 2% w/v saline solution.

14. The method of any one of claims 1-5, wherein the verapamil and mometasone are administered to the subject as a verapamil and mometasone eluting implant placed in the subject's nasal passage or sinuses.

15. The method of claim 14, wherein the implant is bioabsorbable.

16. The method of any one of claims 1-5, wherein the verapamil is administered to the subject's nasal passage and sinuses by nasal irrigation and the mometasone is administered to the subject as a mometasone eluting implant placed in the subject's nasal passage or sinuses.

17. The method of any of the preceding claims, wherein the subject having rhinosinusitis was identified by endoscopy.

18. The method of any of the preceding claims, wherein the subject having rhinosinusitis was identified by computed tomography.

19. The method of any of the preceding claims, wherein the subject having rhinosinusitis was identified by observing the subject's symptoms and duration of symptoms.

20. The method of any of the preceding claims, further comprising monitoring the efficacy of the treatment by endoscopy.

21. The method of any of the preceding claims, further comprising monitoring the efficacy of the treatment by computed tomography.

22. The method of any of the preceding claims, further comprising monitoring the efficacy of the treatment by observing the subject's symptoms and duration of symptoms.

23. The method of any of the preceding claims, further comprising surgically removing any nasal polyps present in the subject.

24. The method of any of the preceding claims, wherein the verapamil and mometasone are administered in combination with an antibiotic.

25. The method of any of the preceding claims, wherein the antibiotic is selected from erythromycin or a pharmaceutically acceptable salt thereof, doxycycline or a pharmaceutically acceptable salt thereof, tetracycline or a pharmaceutically acceptable salt thereof, penicillin or a pharmaceutically acceptable salt thereof, beta-lactam or a pharmaceutically acceptable salt thereof, macrolide or a pharmaceutically acceptable salt thereof, fluoroquinolone or a pharmaceutically acceptable salt thereof, cephalosporin or a pharmaceutically acceptable salt thereof, and sulfonamide or a pharmaceutically acceptable salt thereof.

26. A kit for treating rhinosinusitis in a subject, said kit comprising
a pharmaceutical composition comprising an effective amount of verapamil and mometasone; and
a device for delivering the pharmaceutical composition to the subject's nasal passage and sinuses.

27. The kit of claim 28, wherein said device delivers the pharmaceutical composition to the subject's nasal passage and sinuses in a liquid, nebulized, or aerosolized form.

28. The kit of claim 26 or claim 27, further comprising an antibiotic.

29. A bioresorbable implant comprising verapamil and mometasone.
